(19) United States
(12) Patent Application Publication
(10) Pub. No.: US 2004/0009190 A1
(43) Pub. Date: Jan. 15, 2004

(54) Recombinant attenuation of porcine reproductive and respiratory syndrome (PRRSV)

(75) Inventors: Knut Elbers, Gau-Algesheim (DE); Stefan Pesch, Muenster (DE); Heike Dreier, Coesfeld (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Vetmedica GmbH, Ingelheim (DE)

(21) Appl. No.: 10/600,058

(22) Filed: Jun. 20, 2003

Related U.S. Application Data
(63) Continuation of application No. 09/772,316, filed on Jan. 26, 2001.
(60) Provisional application No. 60/181,605, filed on Feb. 10, 2000.
(60) Provisional application No. 60/181,606, filed on Feb. 10, 2000.
(60) Provisional application No. 60/181,575, filed on Feb. 10, 2000.

(30) Foreign Application Priority Data
Jan. 26, 2000 (DE) 10003371.7-01
Jan. 26, 2000 (DE) 10003372.5
Jan. 26, 2000 (DE) 10003373.3

(51) Int. Cl. A61K 39/12; C07H 21/04; C12N 7/00

(52) U.S. Cl. 424/199.1; 435/235.1; 536/23.72

(57) ABSTRACT

The present invention relates to live PRRS viruses which are attenuated by amino acid mutations in a specific site of the viral protein coded by the open reading frame (ORF) selected from the group of ORF 1a, ORF 1b and/or ORF 2. The invention also pertains to nucleotide sequences coding said viruses, methods of generating such viruses and their use for the preparation of a pharmaceutical composition for the prophylaxis and treatment of PRRS infections.

### RELATED APPLICATIONS

This application claims benefit of prior U.S. application Ser. No. 09/772,316, filed Jan. 26, 2001 which claims benefit of prior provisional application Serial Nos. 60/181575, 60/181605, and 60/181606, all filed Feb. 10, 2000, the disclosures of all of which are incorporated by reference in their entirety.

### FIELD OF THE INVENTION

The present invention relates to live PRRS viruses which are attenuated by amino acid mutations on specific sites of the viral protein coded by the open reading frame (ORF) selected from the group of ORF 1a , ORF 1b and/or ORF 2. The invention also pertains to nucleotide sequences coding said viruses, methods of generating such viruses and their use for the preparation of a pharmaceutical composition for the prophylaxis and treatment of PRRS infections.

### BACKGROUND OF THE INVENTION

Mystery swine disease, later renamed porcine reproductive and respiratory syndrome (PRRS), is caused by an enveloped positive-stranded RNA virus of the family arteriviridae (Snijder E. J. and J. J. M. Meulenberg, 1998, J. Gen. Virol. 79(5):961-971). About 10 to 15 years ago, two different PRRS virus strains emerged apparently independently in the USA and Europe. The disease is now endemic in many swine producing countries in North America, Europe and Asia. It continues to be a major cause of reproductive loss and respiratory disease in swine. In the USA the prevalence of infection is estimated to be up to 70 %.

The virus is transmitted by inhalation, ingestion, coitus, bite wounds or needles. It replicates in mucosal, pulmonary or regional macrophages.

Subclinically, the disease results in resolution or persistent infection. Persistently infected animals shed virus in oral/pharyngeal fluids, blood, feces, urine and semen.

Clinical symptoms in sows relate to abortion or premature farrowing with weak live-born pigs, stillborn pigs and autolyzed fetuses. Infected neonatal pigs have a high mortality or suffer from pneumonia. The subsequent nursery and growth of pigs is complicated by pneumonia, concurrent bacterial infections and increased mortality. Boars are prone to fever and morphological changes in semen.

Like for all arteriviruses, the PRRS virus genome is a single positive-stranded RNA molecule of about 15 kilobases. ORFs (open reading frame) 1a and 1b encode replicases, ORFs 2 to 5 putative glycoproteins (gp 1 to 4), ORF 6 a membrane protein (M) and ORF 7 codes for a nucleocapsid protein (N).

The original descriptions of PRRS infection in the USA (isolated viral agent designated Accession No. ATCC VR-2332, deposited Jul. 18, 1991 at the American Type Culture Collection in Rockville, Md., USA, Genbank U 87392 U00153) and Europe (WO 92/21375, isolate Lelystad Agent (CDI-NL-2.91), deposited Jun. 5, 1991 with the Institute Pasteur, Paris, Accession No. I-1102) identified viruses that had genomic and serological differences. Comparison demonstrated that both had a common ancestor which had diverged before the clinical disease was described in the late 1980's. Full-length genomic sequences have been reported for a number of PRRS viruses and complete structural protein-coding regions thereof (Snijder E. J. and J. J. M. Meulenberg, 1998, J. Gen. Virol. 79(5):961-971; Meulenberg, J. J. M. et al., 1993, Virology 192:62-72; Conzelmann K. K. et al., 1993, Virology 103:329-339; Murtaugh, M. P. et al., 1995, Arch. Virol. 140:1451-1460; Kapur V. et al., 1996, J. Gen. Virol. 77:1271-1276). PRRS virus can be replicated in vitro in pig lung macrophages, monocytes, glial cells and two MA- 104 cell subpopulations (embryonic monkey kidney cell) known as CL-2621 and MARC-145 (Rossow, K. D., 1998, Vet. Pathol 35:1-20). Recombinant means for generating infectious PRRS clones are also available (EP 0 839 912 A1).

For protecting pigs, live attenuated (e.g., Ingelvac® PRRS MLV, Boehringer Ingelheim) PRRS vaccines are commercially available. The Ingelvac® PRRS MLV vaccine comprises passage 70 of ATCC VR-2332 which was deposited with the American Tissue Culture Collection under Accession No. ATCC VR-2495.

Killed vaccines (inactivated whole virus) or subunit vaccines (conventionally purified or heterologously expressed purified viral proteins) are most often inferior to live vaccines in their efficacy to produce a full protective immune response even in the presence of adjuvants. For PRRS it has been demonstrated, that in comparison to the currently available killed vaccines, the attenuated vaccines induce an immunity against the disease which lasts longer and is more efficient (Snijder et al., referenced above). The present live PRRS vaccines are attenuated conventionally by serially passaging the virus in appropriate host cells until pathogenicity is lost (EP 0529584 B1). Present live PRRS vaccines still leave ample room for improvement. For one, they do not prevent reinfection. Secondly, they do not allow serological discrimination between vaccinated animals and animals infected with the field virus. But most important of all, live vaccines from complete microorganisms, although attenuated, can be associated with serious safety problems. This holds especially true for RNA viruses such as the PRRS virus, which are considered to have high rates of mutation due to imprecise replication of the RNA genome resulting from a lack of proofreading by the RNA replication enzyme.

A potential reversion of attenuated live viruses can pose a serious threat to vaccinated animals. For conventionally derived attenuated viruses wherein the attenuation is attained by conventional multiple passaging, the molecular origin as well as the genetic stability remains unknown and the outbreak of revertants is unpredictable.

Therefore, the technical problem underlying this invention was to provide PRRS viruses less likely to revert to wild type viruses.

### SUMMARY OF THE INVENTION

The present invention relates to live PRRS viruses which are attenuated by amino acid mutations on specific sites of the viral protein encoded by the open reading frame (ORF) selected from the group of ORF 1a, ORF 1b and/or ORF 2. The invention also pertains to nucleotide sequences encoding said viruses, methods of generating such viruses and their use for the preparation of a pharmaceutical composition for the prophylaxis and treatment of PRRS infections.

### BRIEF DESCRIPTION OF THE FIGURES

FIG. 1: Amino acid sequence of ORF 1a of ATCC VR-2332 (SEQ ID NO:1) with preferred attenuation sites according to the invention marked.

FIG. 2: Amino acid sequence of ORF 1b of ATCC VR-2332 (SEQ ID NO:2) with preferred attenuation sites according to the invention marked.

FIG. 3: Amino acid sequence of ORF 2 of ATCC VR-2332 (SEQ ID NO:3) with preferred attenuation sites according to the invention marked.

FIG. 4: Nucleotide sequence of ORF 1a of ATCC VR-2332 (SEQ ID NO:4) with preferred attenuation sites according to the invention in bold and most preferred sites underlined.

FIG. 5: Nucleotide sequence of ORF 1b of ATCC VR-2332 (SEQ ID NO:5) with preferred attenuation sites according to the invention in bold and most preferred sites underlined.

FIG. 6: Nucleotide sequence of ORF 2 of ATCC VR-2332 (SEQ ID NO:6) with preferred attenuation sites according to the invention in bold and most preferred sites underlined.

### DETAILED DESCRIPTION OF THE INVENTION

The solution to the above technical problem is achieved by the description and the embodiments characterized in the claims.

Before the embodiments of the present invention it must be noted that as used herein and in the appended claims, the singular forms “a”, “an”, and “the” include plural reference unless the context clearly dictates otherwise. Thus, for example, reference to “a PRRS virus” includes a plurality of such PRRS viruses, reference to the “cell” is a reference to one or more cells and equivalents thereof known to those skilled in the art, and so forth. Unless defined otherwise, all technical and scientific terms used herein have the same meanings as commonly understood by one of ordinary skill in the art to which this invention belongs.

Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

It has surprisingly been found that PRRS viruses comprise specific genomic sites in some open reading frames that consistently revert to the amino acids encoded by ATCC VR-2332 at that position. The evolutionary pressure on this from now on called “virulence specific site” or simply referred to as “site of the invention or just “site” is immense. For two revertant strains of ATCC VR-2495, it was possible to demonstrate for the first time that the amino acid mutation to the amino acid of ATCC VR-2332 at this virulence specific site occurred geographically independently in both, the USA and Europe.

Live vaccines with defined mutations as a basis for attenuation according to the invention avoid the disadvantages of the present generation of attenuated vaccines. A further advantage of said attenuating mutations lies in their known molecular uniqueness which allows for use as distinctive labels for attenuated pestiviruses and to distinguish them from pestiviruses from the field.

The amino acid and nucleotide sequence of the conventionally attenuated virus ATCC VR-2495 were compared to ATCC VR-2332. To identify the virulence specific sites, the two mentioned virulent revertants were compared to each other and ATCC VR-2332 as well as ATCC VR-2495.

This allowed for the identification of the virulence specific site on an individual viral protein that is implicated in the virulence of PRRS viruses.

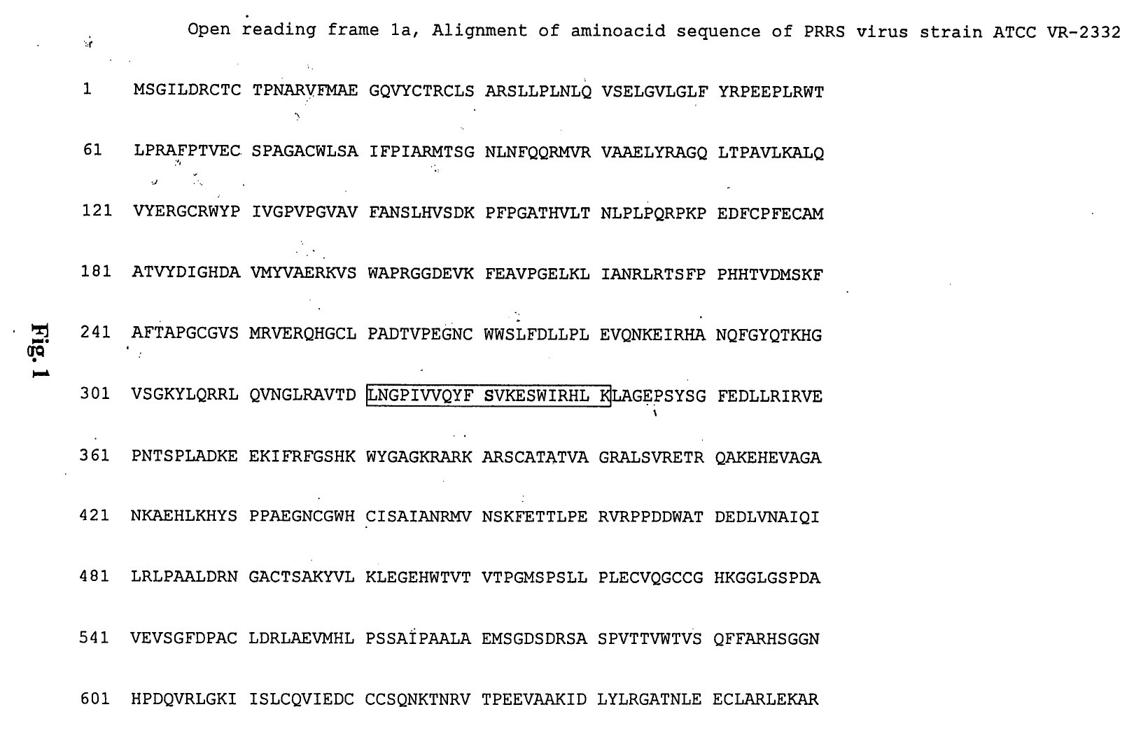

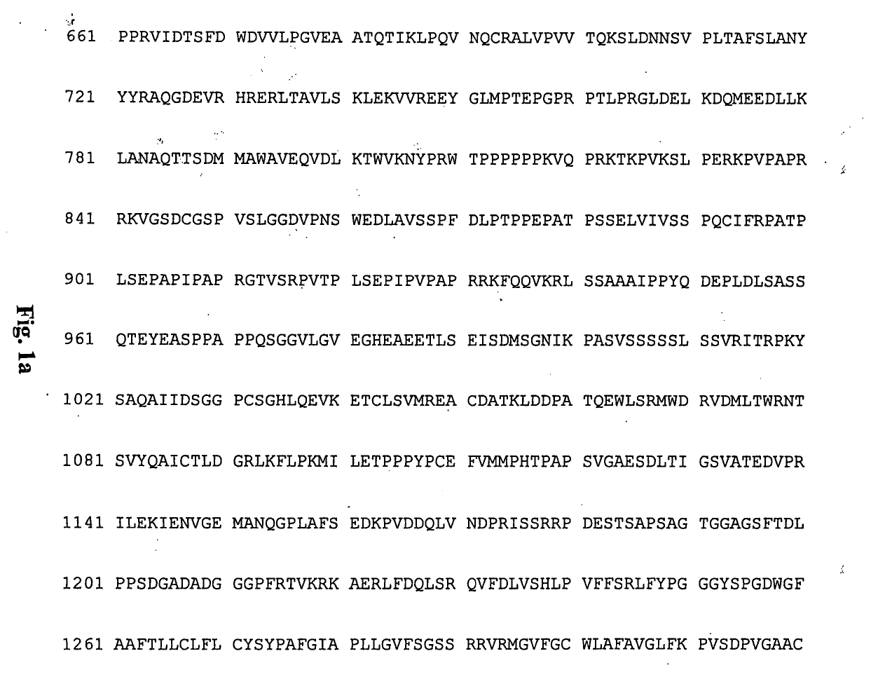

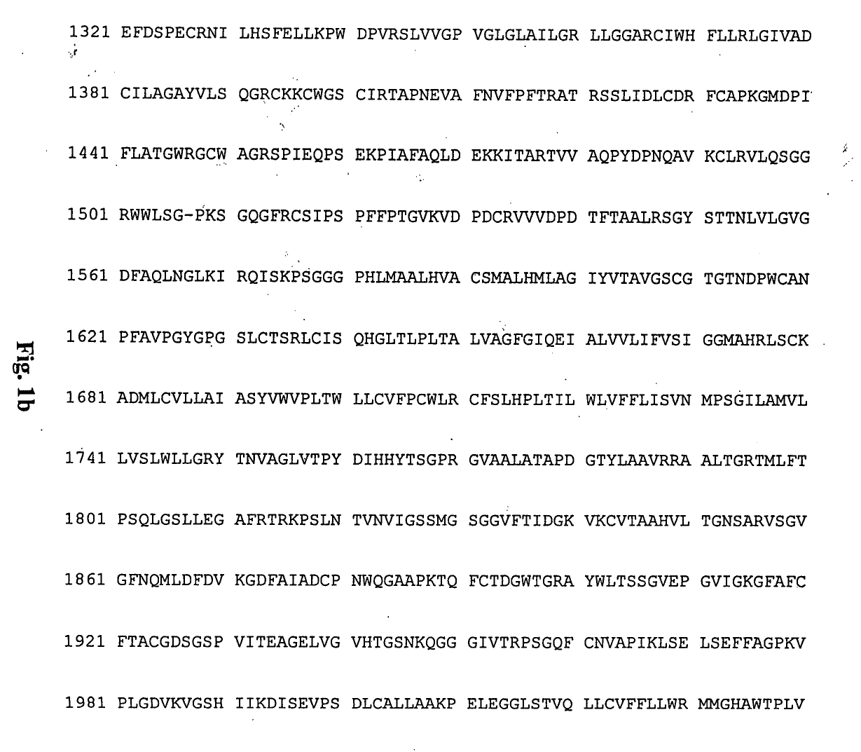

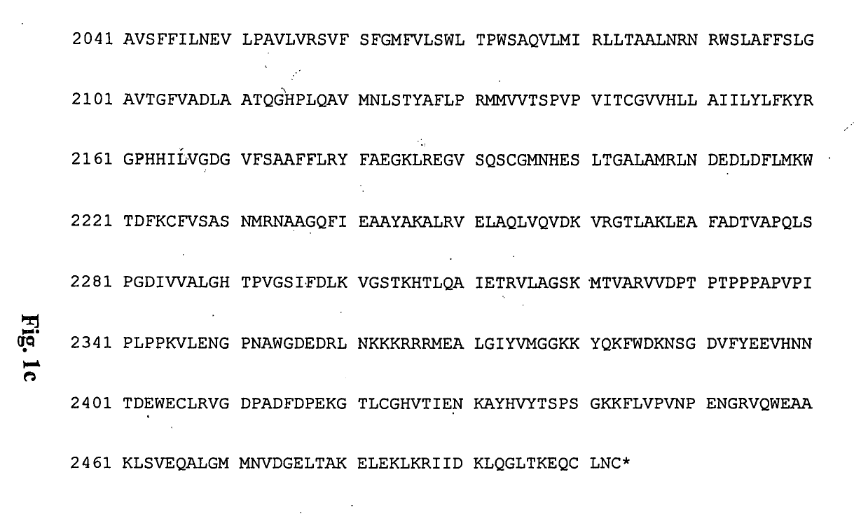

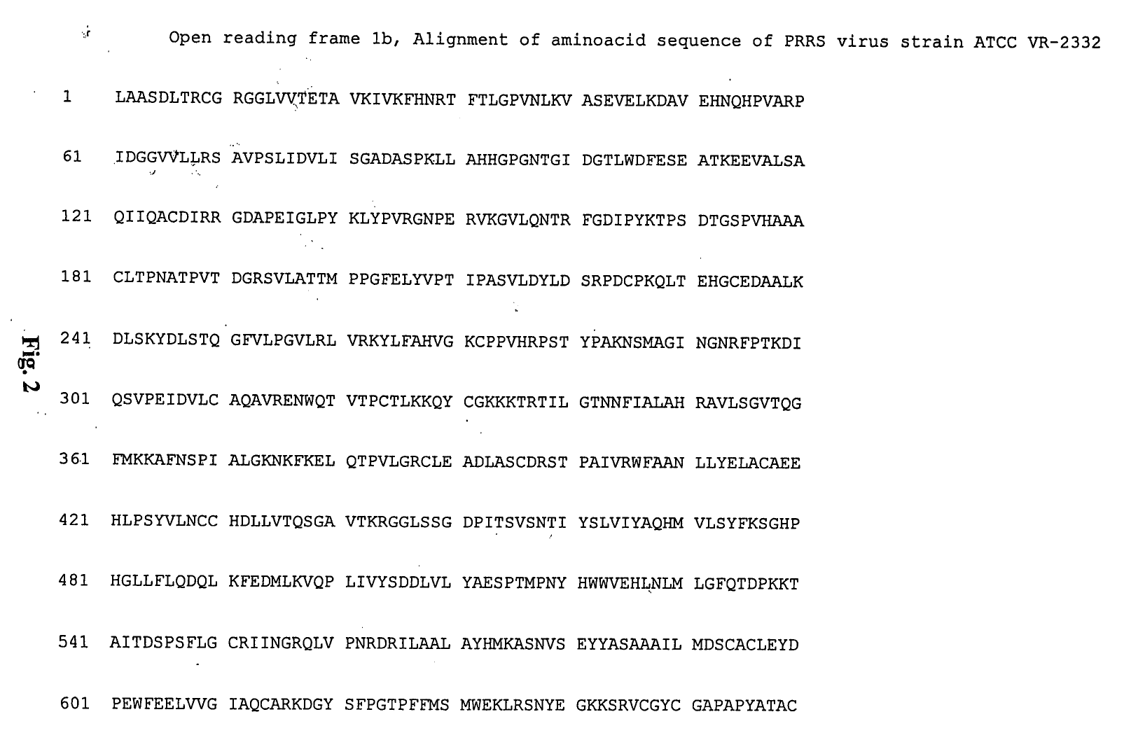

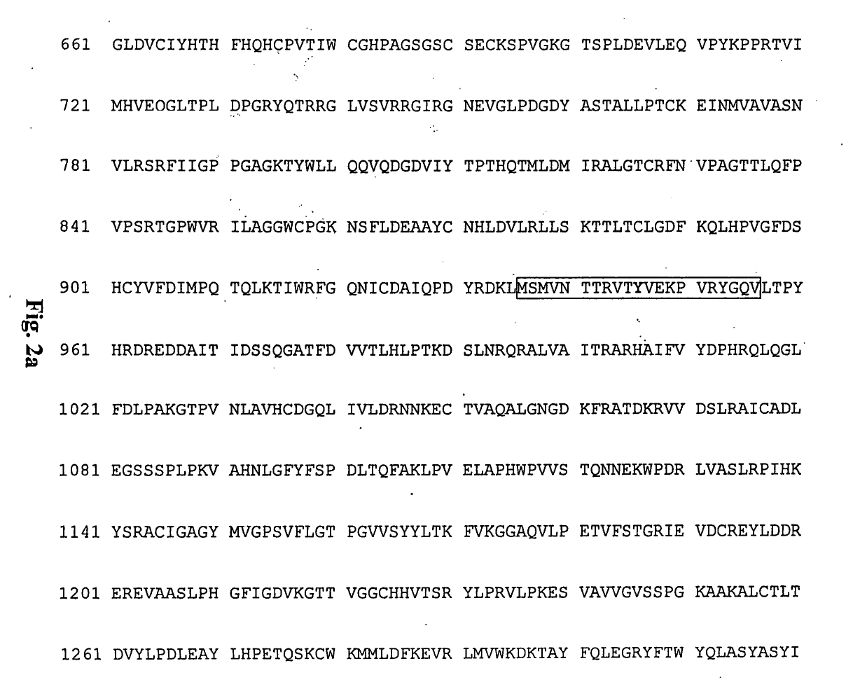

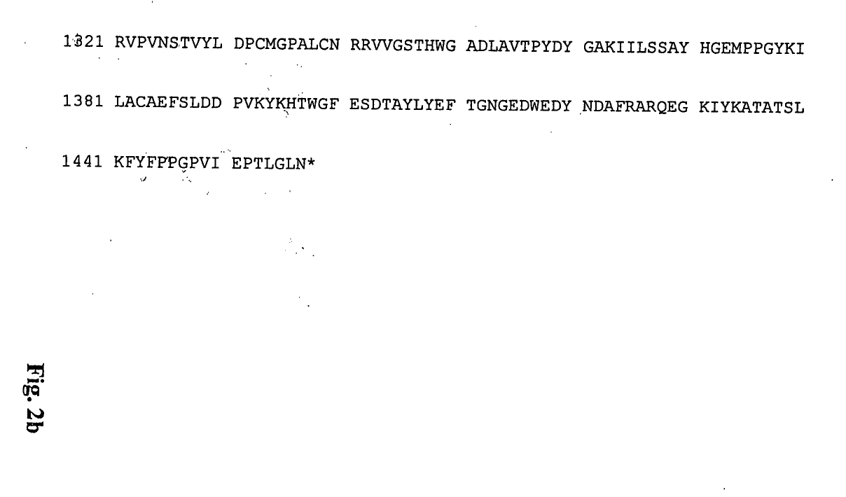

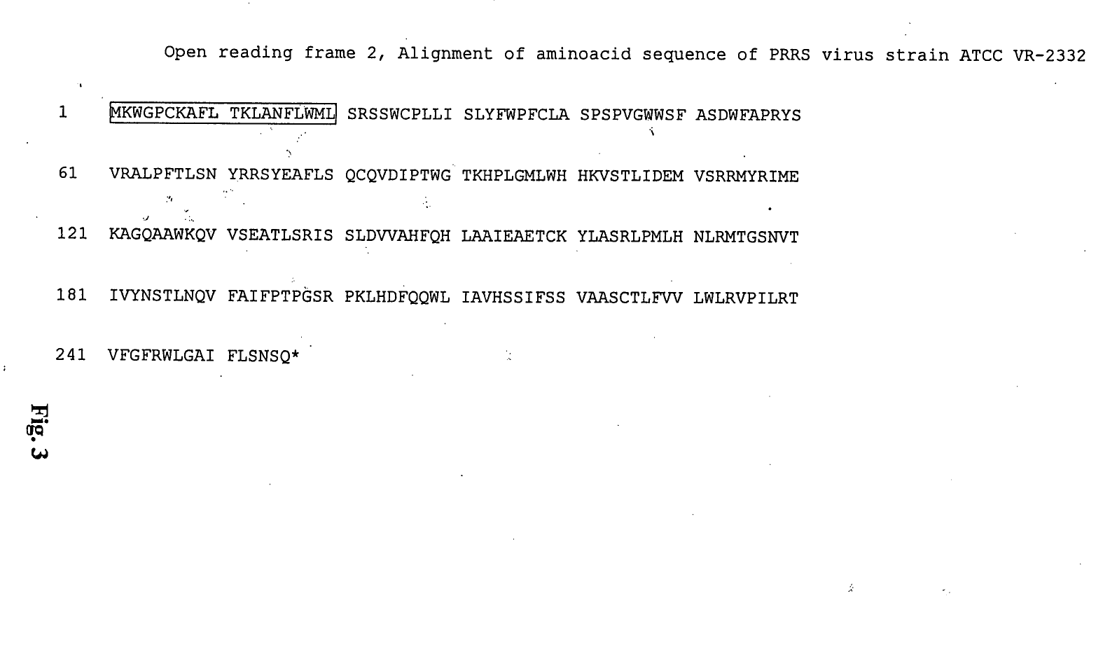

In consequence, one aspect of the invention relates to live PRRS viruses which are not ATCC VR-2495 and which are less virulent than the PRRS virus ATCC VR-2332 that are characterized in that they comprise a protein encoded by the open reading frame (ORF) selected from the group of ORF 1a as described in FIG. 1 for said ATCC VR-2332 strain, ORF 1b as described in FIG. 2 for said ATCC VR-2332 strain, and/or ORF 2 as described in FIG. 3 for said ATCC VR-2332 strain, wherein at least one of the amino acids at the identified virulence specific sites is not identical to at least one of the amino acids of the strain ATCC VR-2332 at said corresponding positions.

The numbering of amino acids and nucleotides (nt) is according to the database entry of VR-2332. FIGS. 1, 2 and 3 provide information that is representative for all PRRS strains and allows visualization of the invention and identification of the preferred amino acids and preferred site in all PRRS viruses that might be numbered differently. Identification of these positions is achieved by identifying preserved characteristic identical amino acids in a PRRS strain of interest and the listed reference strain and subsequently determining the position of the site of the virus of interest relative to the site in FIG. 1, 2 or 3.

Three of said sites have been identified for the protein encoded by the viral ORF 1a, ORF 1b and ORF 2 which are depicted for ATCC VR-2332 in FIGS. 1, 2 and 3, respectively.

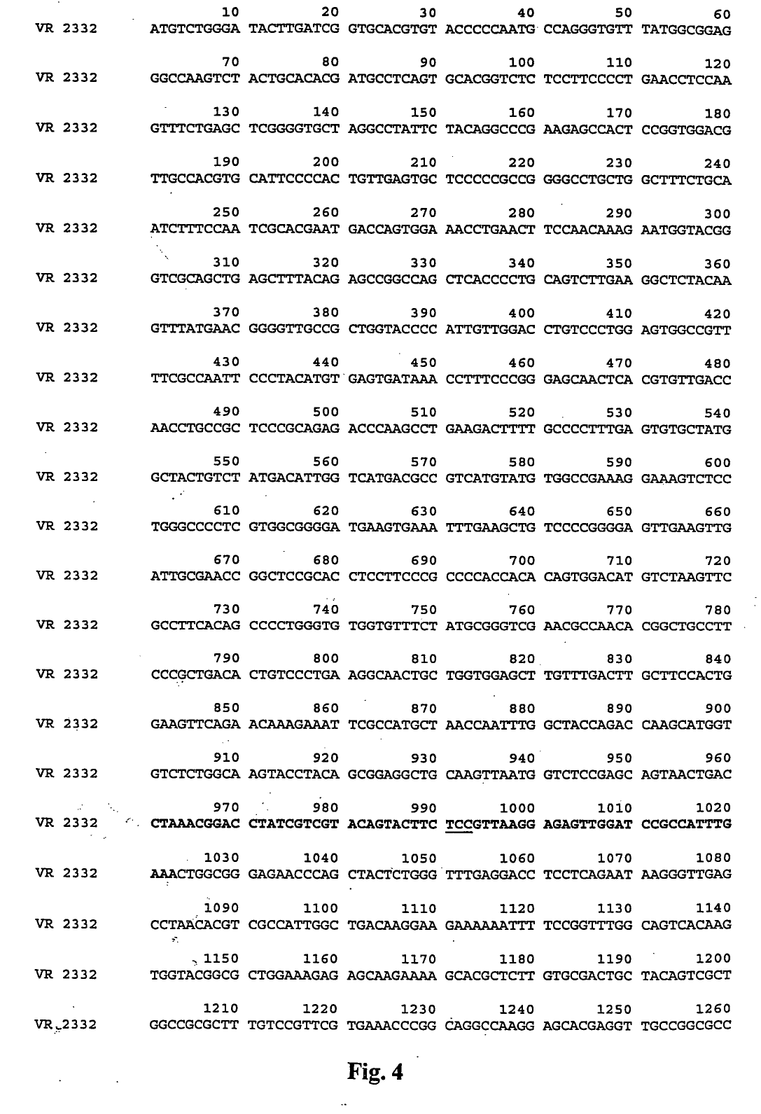

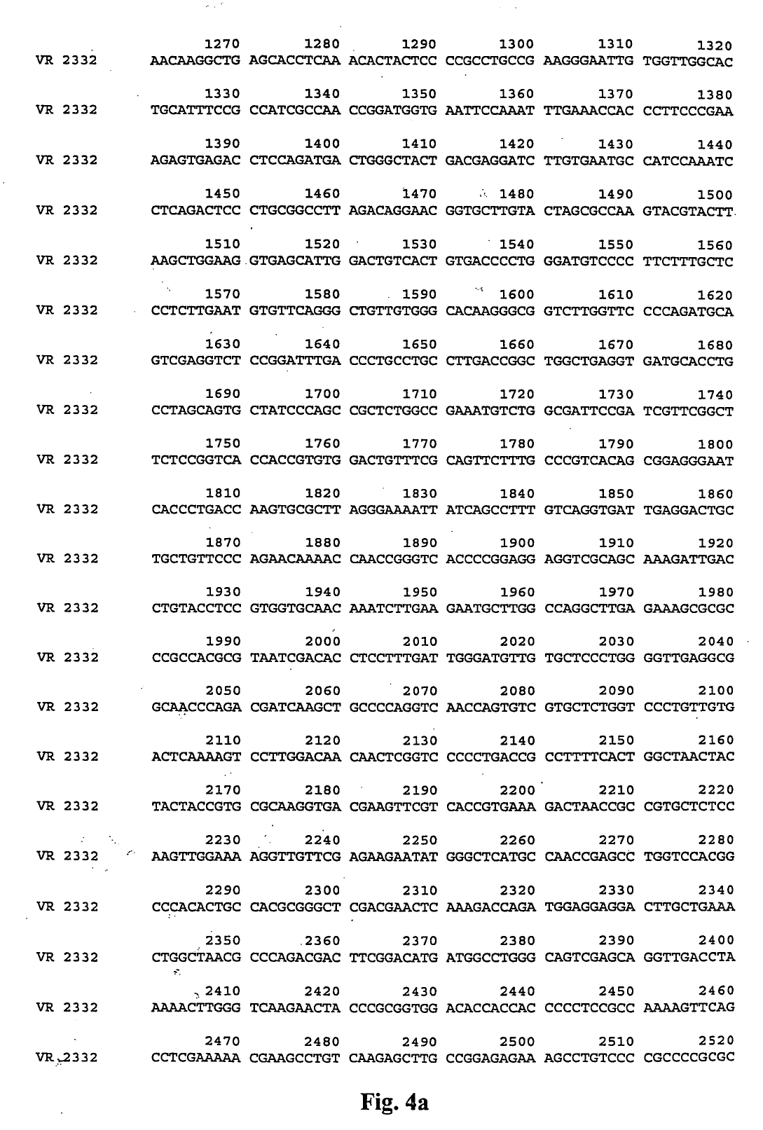

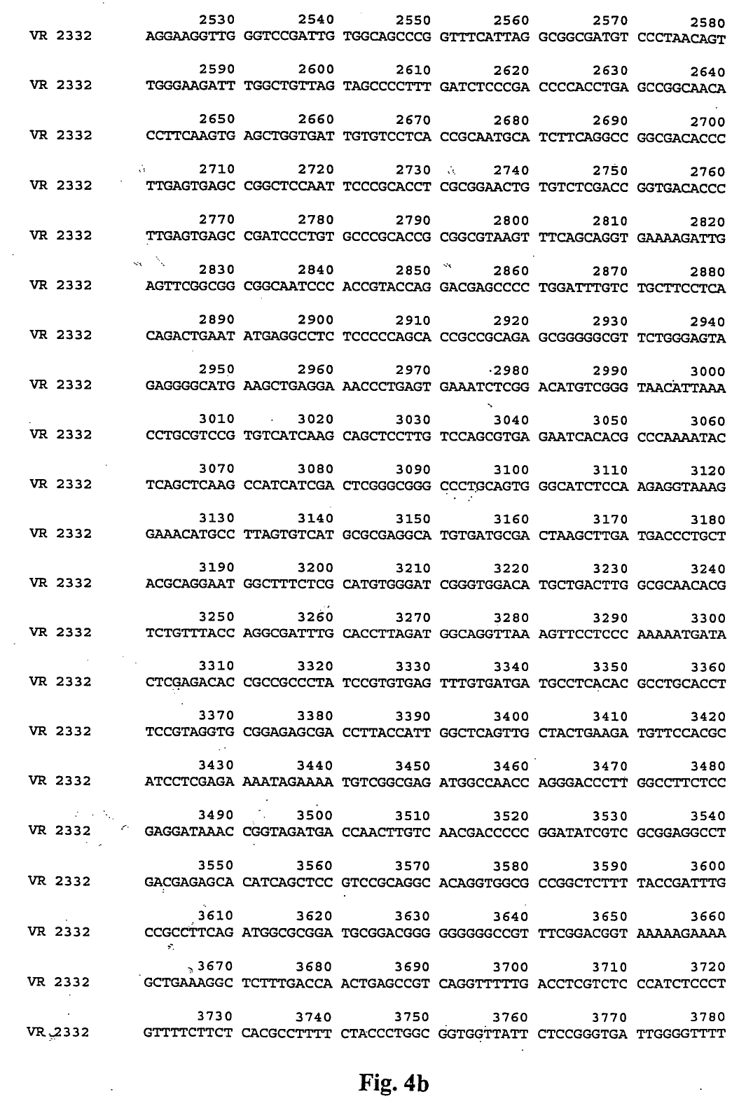

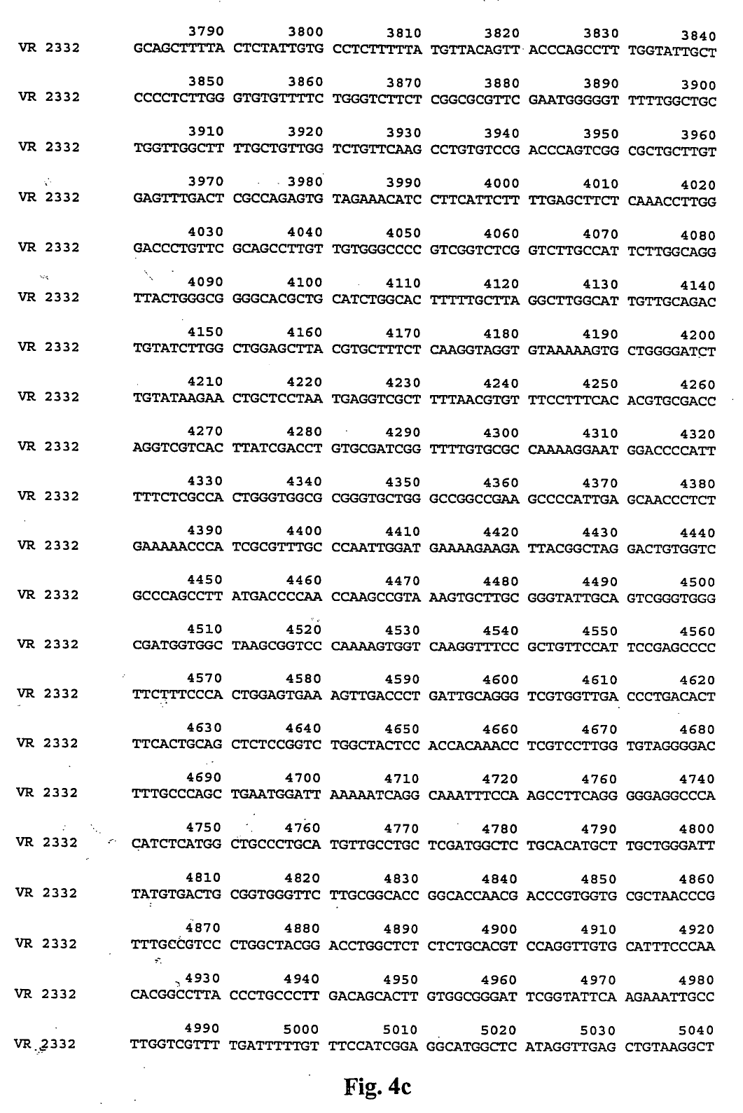

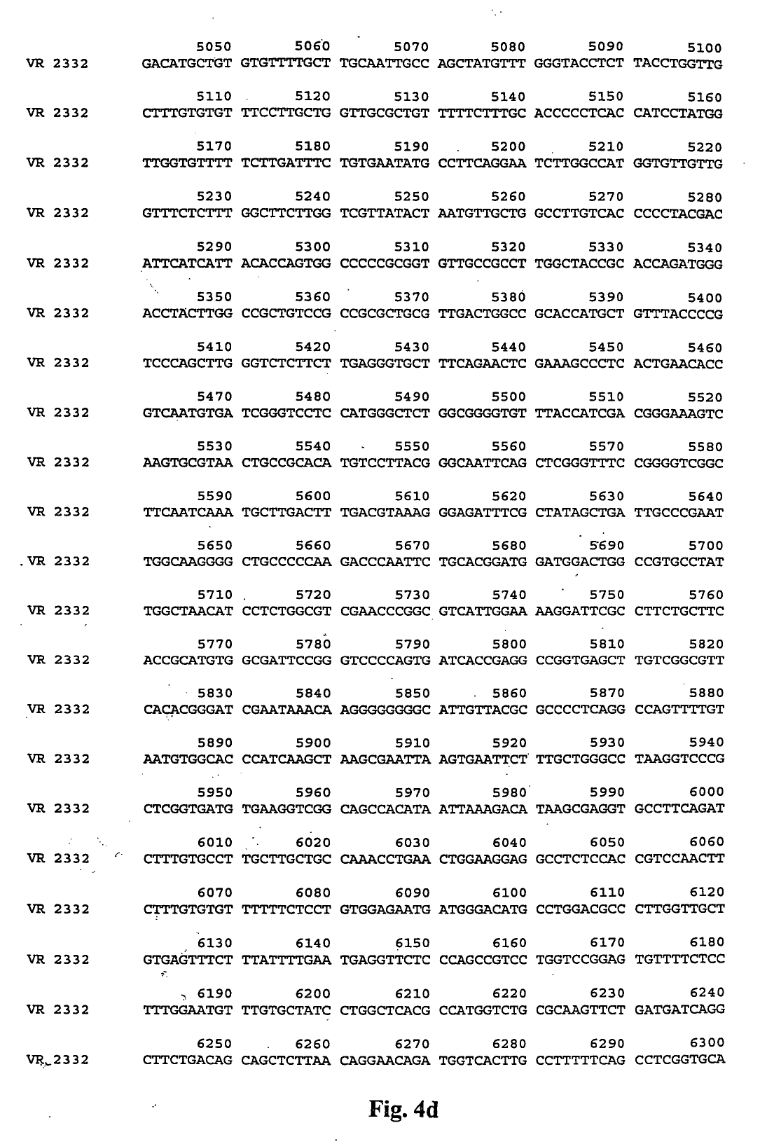

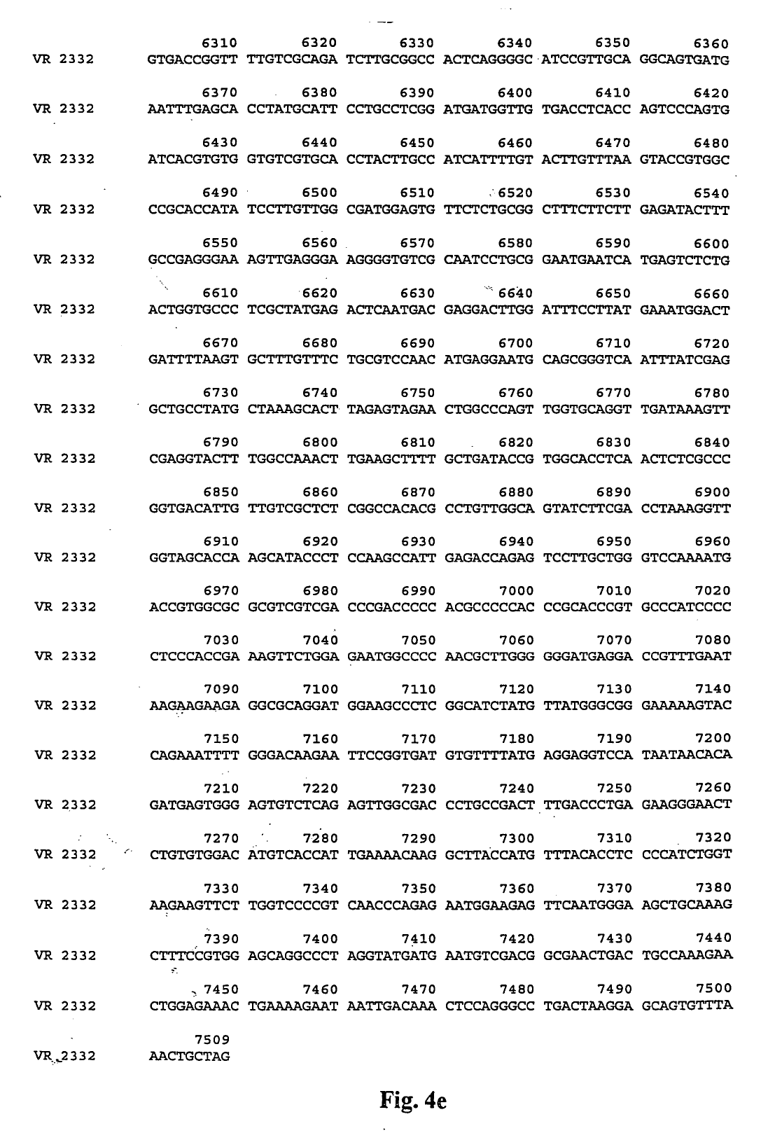

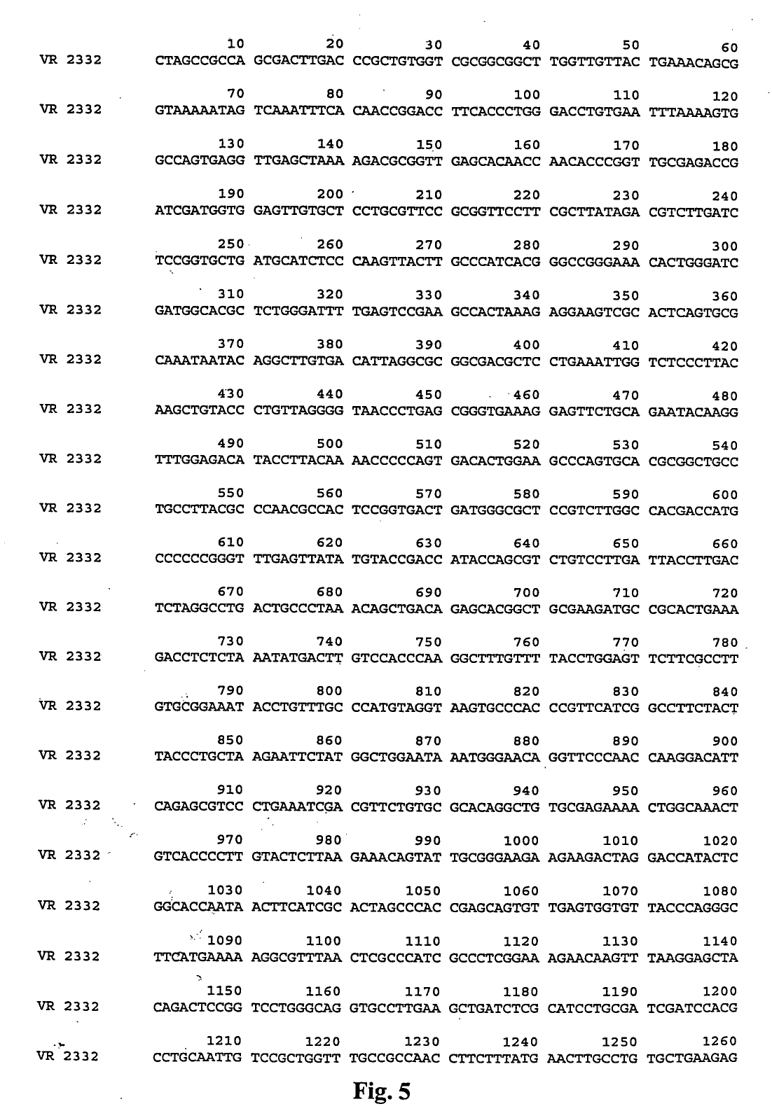

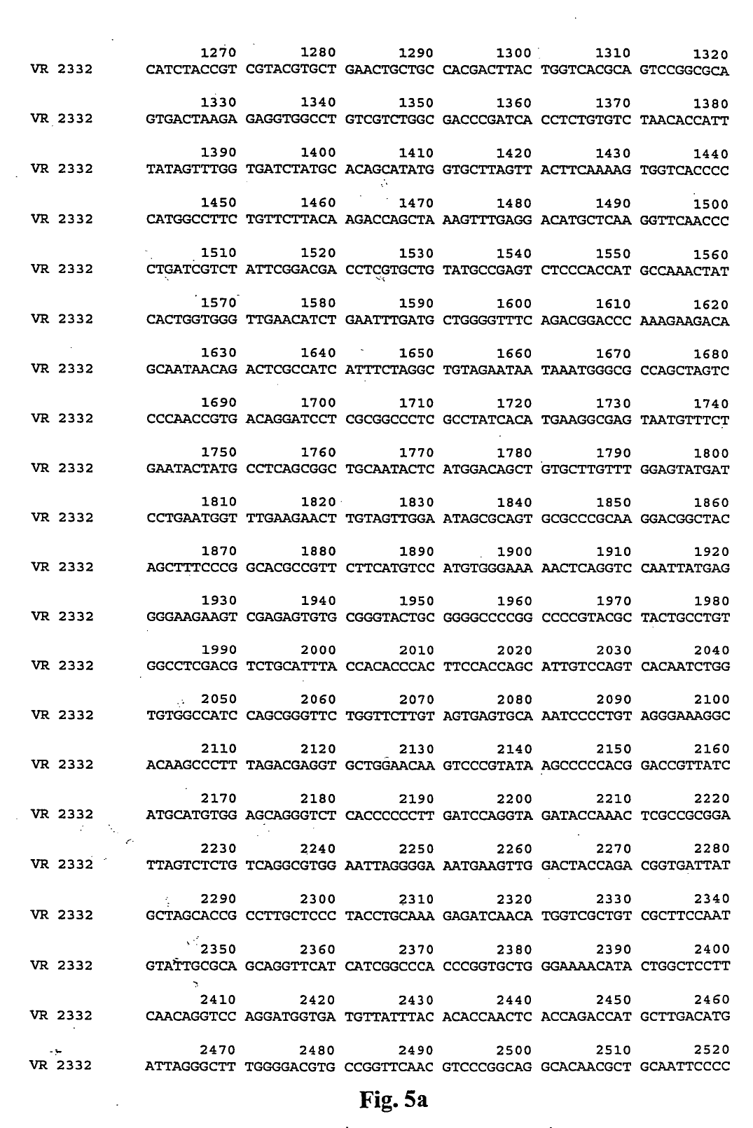

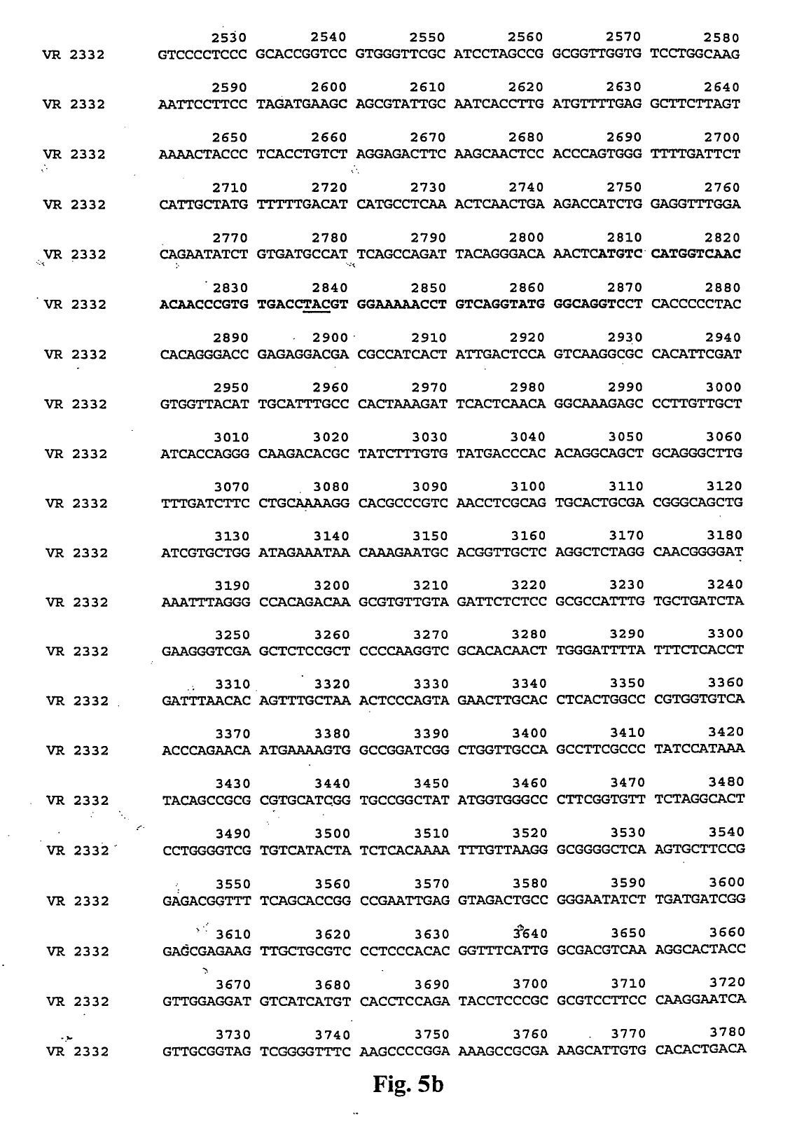

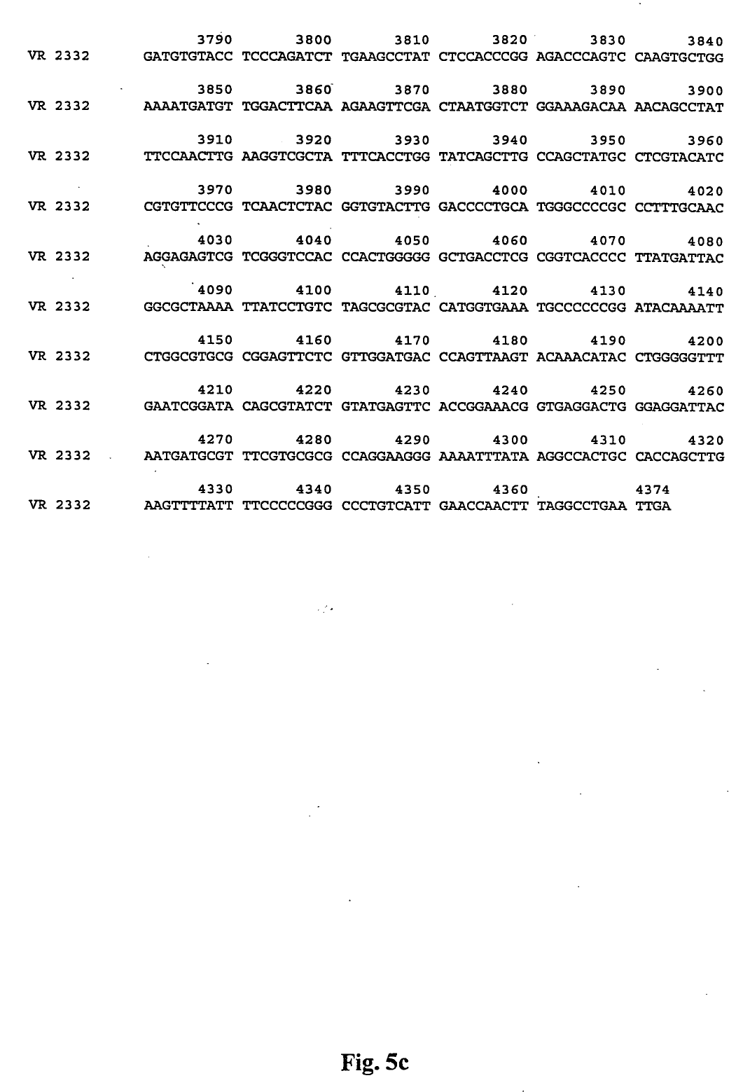

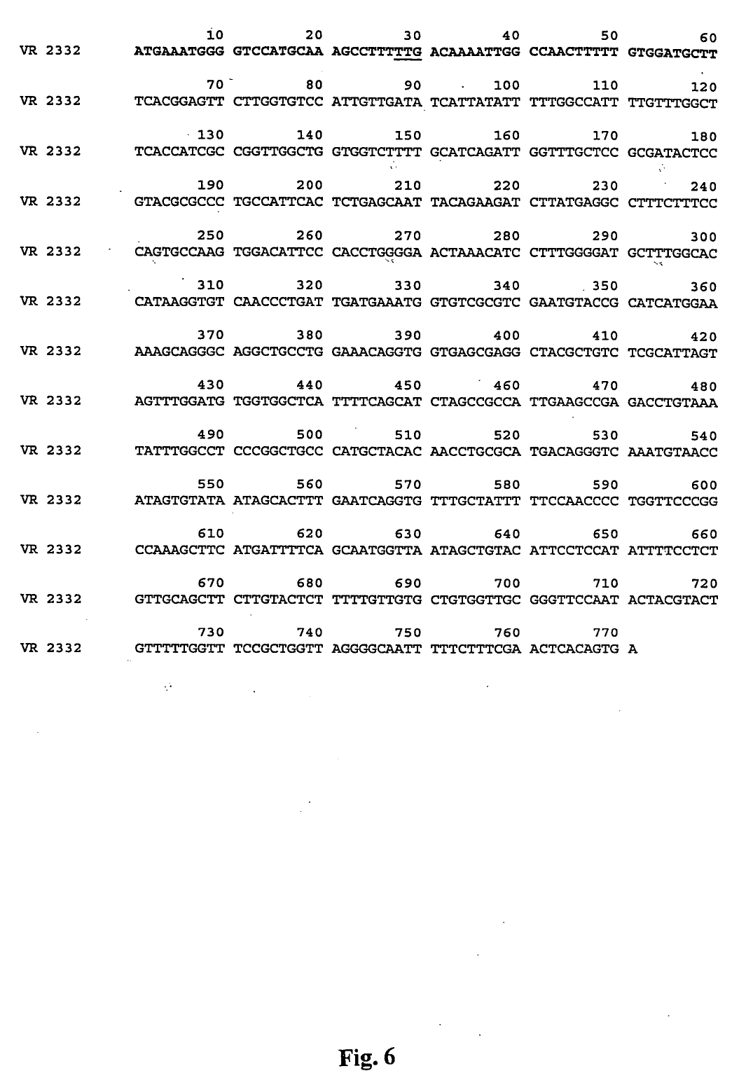

Another aspect of the invention relates to a live attenuated PRRS virus comprising ORF 1a, ORF 1b and ORF 2 essentially as in ATCC VR-2332 which is not ATCC VR-2495, characterized in that at least one of the amino acids in position 321 to 341 of the protein encoded by ORF 1a is not identical to the amino acid(s) of the strain ATCC VR-2332 at said corresponding position(s) as described in FIG. 1 and/or at least one of the amino acids in position 936 to 956 of the protein encoded by ORF 1b is not identical to the amino acid(s) of the strain ATCC VR-2332 at said corresponding position(s) as described in FIG. 2 and/or at least one of the amino acids in position 1 to 20 of the protein encoded by ORF 2 is not identical to the amino acid(s) of the strain ATCC VR-2332 at said corresponding position(s) as described in FIG. 3. Thus, at the amino acid site 321-341 encoded by ORF 1a (nt 961-1023), amino acid site 936-956 encoded by ORF 1b (nt 2806-2868), or amino acid site 1-20 encoded ORF 2 (nt 1-60) (See FIG. 4, 5 or 6, respectively), the coding nucleotide triplet for one amino acid, or for more than one amino acids is/are mutated resulting in one or several changes in the sequence at said site either on the nucleic acid level or in addition and preferentially also on the amino acid level, whereby up to all nucleotide or amino acid positions at a said site may be mutated. Viruses where either one, two, or all three of said sites are mutated are embraced by the present invention.

“Mutation” means the replacement of an amino acid for another or the replacement of the coding nucleotide by another (e.g. C for a T), i.e., a so-called “substitution”, preferably in a way that the encoded amino acid is changed, or any other mutation such as “deletion” or “insertion”. The mutation is always carried out in the coding nucleotide sequence.

Said mutations may be carried out by standard methods known in the art, e.g. site directed mutagenesis (see e.g. Sambrook et al.(1989) Molecular Cloning: A Laboratory Manual, 2^{nd} ed., Cold Spring Harbor Laboratory Press, Cold Spring Harbor, N.Y.) of an infectious copy as described (e.g. Meulenberg et al., Adv. Exp. Med. Biol, 1998, 440:199-206).

“Essentially” means at least 75% of the sequence, preferably 85%, most preferably all of the sequence except the “sites” according to the invention is identical to ATCC VR-2332. However, additional nucleic acids coding for amino acids outside said sites according to the invention may also be mutated. Said virus according to the invention is still fulfilling the criteria according to the invention, i.e., being less likely to revert to wild type and also less virulent than ATCC VR-2332.

A live PRRS virus according to the invention refers to a PRRS virus as defined in Snijder et al. (referenced above) that is capable of infecting swine and capable of replication in swine.

The conventionally attenuated ATCC VR-2495 virus is specifically disclaimed. It is not a virus according to the invention and is specifically excluded from the scope of the claims. The vaccine comprising ATCC VR-2495 virus is commercially available from the Boehringer Ingelheim Vetmedica company (Ingelvac® PRRS MLV). Most of its sequence is publicly available (Genbank AJ 223082).

The site and amino acids of particular importance to the invention are by no means limited to the exact position as defined for the ATCC VR-2332 strain but are simply used in an exemplary manner to point out the preferred amino acids being at that position or corresponding to that position in other PRRS strains. For different PRRS viruses the numbering of the positions of the preferred amino acids might be different but an expert in the field of the molecular biology of viruses of the family arteriviridae will easily identify these preferred amino acids by their position relative to the other conserved amino acids of said proteins.

The term “less virulent than the PRRS virus ATCC VR-2332” is to be understood in terms of a comparison of clinical symptoms of the virus of interest with ATCC VR-2332. A preferred procedure for determining if a PRRS virus is less virulent than the PRRS virus ATCC VR-2332 is listed in Example 1. Not all possible preferred amino acid mutations at the virulence specific site might be implicated in reducing virulence. The procedure of Example 1 provides a precise and straight forward experimental setup for determining whether a live PRRS virus, that comprises the protein according to the teaching of the invention, is less virulent than ATCC VR-2332.

The virulence specific site was identified by the reversion of at least one amino acid from the attenuated virus to the amino acid of ATCC VR-2332. This particular amino acid is part of a larger secondary peptide structure such as an alpha helix or a β sheet or a hairpin β motif or others. It is therefore highly probable that neighboring amino acids are also involved in the regulation of virulence of that protein. An expert in the field of protein chemistry would therefore expect a high probability of identifying further amino acids with virulence-implicated properties within the vicinity of 10 amino acids to the left and right of the originally identified amino acid position. Ten to 20 amino acids is the typical range for peptide motifs in proteins. Therefore, preferred viruses according to the invention comprise nucleotides encoding the protein described in FIG. 1 in an exemplary manner or corresponding thereto in other strains, wherein the virulence specific site comprises 10 amino acids upstream and 10 amino acids downstream of the originally identified amino acid position. More preferred are those viruses as mentioned above, wherein the virulence specific site comprises 5 amino acids upstream and 5 amino acids downstream of the originally identified amino acid position. Most preferred are those viruses as mentioned above, wherein the virulence specific site comprises 3 amino acids upstream and 3 amino acids downstream of the originally identified amino acid position.

With the teaching of the present invention, it is possible to generate attenuated PRRS strains from virulent strains by mutating the nucleotides encoding amino acids at the virulence specific site. Still, the safety problem associated with the high frequency of mutation in RNA viruses remains. This problem can be greatly reduced by deleting specific amino acids in the virulence specific site of the virus protein. The invention, therefore, relates to PRRS viruses as mentioned above that are characterized in that at least one of the amino acids in the virulence specific site of the viral protein is deleted. The term “deleted” is to be understood as being absent in comparison to the amino acids referenced in the figure at that or those position(s). Thus, according to the invention, “deletion” means the removal of one or several nucleotides or amino acids.

In a more preferred embodiment the invention therefore relates to a live attenuated PRRS virus according to the invention, characterized in that at least one of the amino acids in position 321 to 341 of the protein encoded by ORF 1a is deleted and/or at least one of the amino acids in position 936 to 956 of the protein encoded by ORF 1b is deleted and/or at least one of the amino acids in position 1 to 20 the protein encoded by ORF 2 is deleted. Thus, either at the amino acid site 321-341 encoded by ORF 1a , amino acid site 936-956 encoded by ORF 1b , or amino acid site 1-20 encoded by ORF 2 or at all three sites the coding nucleotide triplet for one amino acid, or for more than one amino acids is/are mutated resulting in one or several changes in the sequence at said site either on the nucleic acid level or in addition and preferentially also on the amino acid level, whereby up to all nucleotide or amino acid positions at a said site may be mutated. Viruses where either one, two, or all three of said sites are mutated are embraced by the present invention.

For the identified virulence specific site on the particular PRRS virus protein it has been demonstrated in a preferred example that at least one amino acid is under high mutational pressure and involved in the virulent properties of revertants of ATCC VR-2332. This individual amino acid position is a most preferred embodiment of the invention.

Therefore, in a most preferred embodiment, the present invention relates to a live attenuated PRRS virus according to the invention which is not ATCC VR-2495 and which is less virulent than ATCC VR-2332, characterized in that the amino acid in position 331 of the protein encoded by ORF 1a and/or the amino acid in position 946 of the protein encoded by ORF 1b and/or the amino acid in position 10 of the protein encoded by ORF 2 is/are not identical to the amino acid of the strain ATCC VR-2332 at said corresponding position. Thus, either at the site 331 of ORF 1a , site 946 of ORF 1b , or site 10 of ORF 2 or at all three sites the coding nucleotide triplet for one amino acid or the amino acid is mutated resulting in one to three mutations at said site(s) (See FIG. 4, 5 or 6, respectively).

For the reasons presented above, it is safer and preferable to avoid the reversion of altered amino acids to the virulent type by simply deleting the amino acid that is prone to revert.

Therefore, the present invention relates in this most preferred embodiment to a live attenuated PRRS virus according to the invention, characterized in that the amino acid in position 331 of the protein encoded by ORF 1a and/or the amino acid in position 946 of the protein encoded by ORF 1b and/or the amino acid in position 10 of the protein encoded by ORF 2 is/are deleted, in other words, absent when compared to that position in ATCC VR-2332.

A further, most preferred embodiment is a live attenuated PRRS virus according to the invention, characterized in that the amino acid in position 331 of the protein encoded by ORF 1a , i.e., the coding triplet is deleted. A further, most preferred embodiment is a live attenuated PRRS virus according to the invention, characterized in that the amino acid in position 946 of the protein encoded by ORF 1b , i.e., the coding triplet is deleted. A further, most preferred embodiment is a live attenuated PRRS virus according to the invention, characterized in that the amino acid in position 10 of the protein encoded by ORF 2, i.e., the coding triplet is deleted (See FIG. 4, 5 or 6, respectively). Said most preferred virus is identical to ATCC VR-2332 in all other positions.

The teaching of the present invention now enables the expert to recombinantly produce infectious clones of PRRS viruses (PRRSV) that are less virulent than ATCC VR-2332 and are useful for preparing a live pharmaceutical composition. All information required to produce recombinant infectious clones of positive strand RNA viruses is readily available in the art, particularly for the PRRSV. For example, the European patent application EP 0 839 912 of Meulenberg et al., which is referenced herewith in its entirety, provides a clear teaching for the preparation of recombinant live PRRS viruses. Therefore, in a further aspect, the present invention relates to nucleotide sequences coding for a virus according to the invention. Due to the degeneration of the genetic code multiple nucleotide variants may result in the identical amino acid translation. Those degenerate variants are also encompassed by the invention.

As mentioned in the introductory pages, it is important for the health management of pigs to be able to distinguish between the less virulent live vaccine strain of the pharmaceutical composition and the virulent wild type virus infections. This is often difficult, especially when clinical symptoms of a field infection are not that specific or superimposed by other infections or the time period for observation and evaluation is short. The recombinant generation of the viruses of interest allows for the introduction of modifications in the genetic code that establishes a serological marker and/or a virulence marker. A serological marker refers to an antigenically detectable molecule such as a peptide, a protein, glycoprotein that can be isolated from infected cells or body fluids such as but not limited to pharyngeal or nasal fluids or urine. A virulence marker is to be understood as a marker in the genetic code that can be identified by recombinant analytical methods such as but not limited to PCR and conventional sequencing. Therefore, in a preferred embodiment, the present invention relates to a nucleotide sequence according to the invention, wherein the nucleotide sequence has been modified to encode a virulence marker and/or a serological marker. Particularly, the mutations or deletions introduced for the purpose of attenuating virulence are useful as virulence and serological markers. By monitoring these mutations in the disclosed virulence specific sites it is possible to predict the emergence of possibly virulent revertants at an early stage.

It is more preferred that the nucleotide sequences of the invention are such that the nucleotide sequence encoding said marker is located within any of the open reading frames encoding structural viral proteins.

A further aspect of the present invention relates to a method for the generation of an infectious live attenuated PRRS virus according to the invention, said method comprising producing a recombinant nucleic acid as described above comprising at least one full-length DNA copy or in vitro-transcribed RNA copy or a derivative of either.

Another preferred embodiment according to the invention relates to a method according to the invention, wherein specific mutations are inserted with molecular biology methods, characterized in that the nucleic acid corresponding to amino acid positions 321 to 341 of ORF 1a and/or the nucleic acid corresponding to amino acid positions 936 to 956 of ORF and/or the nucleic acid corresponding to amino acid positions 1 to 20 of ORF 2 is mutated in such a way that at least one nucleotide at said positions is substituted or deleted.

Another important aspect of the invention is a pharmaceutical composition comprising a PRRS virus according to the invention and a pharmaceutically acceptable carrier.

A “pharmaceutical composition” essentially consists of one or more ingredients capable of modifying physiological e.g. immunological functions of the organism it is administered to or of organisms living in or on its surface including but not restricted to antibiotics or antiparasitics, as well as other constituents added to it in order to achieve certain other objectives including, but not limited to, processing traits, sterility, stability, feasibility to administer the composition via enteral or parenteral routes such as oral, intranasal, intravenous, intramuscular, subcutaneous, intradermal or other suitable route, tolerance after administration, controlled release properties.

A pharmaceutically acceptable carrier can contain physiologically acceptable compounds that act, for example, to stabilize or to increase the absorption or form part of a slow release formulation of the PRRS virus according to the invention. Such physiologically acceptable compounds include, for example, carbohydrates, such as glucose, sucrose or dextrans, antioxidants, such as ascorbic acid or glutathione, chelating agents, low molecular weight proteins or other stabilizers or excipients (see also e.g. Remington's Pharmaceutical Sciences (1990). 18th ed. Mack Publ., Easton). One skilled in the art would know that the choice of a pharmaceutically acceptable carrier, including a physiologically acceptable compound, depends, for example, on the route of administration of the composition.

A further aspect of the invention relates to the use of the viruses according to the invention. With the availability of the live attenuated PRRS viruses of the invention it is now possible to use these in the manufacture of a vaccine for the prophylaxis and treatment of PRRS infections. Their defined molecular basis of attenuation makes them superior to the present conventionally attenuated viruses. Especially the use of viruses according to the invention that comprise deletions in the virulence specific sites is preferred since deletions are less prone to revert.

The following example serves to further illustrate the present invention; but the same should not be construed as limiting the scope of the invention disclosed herein.

### EXAMPLE 1

Establishment of Attenuation

This example provides a clear guidance for the comparison of the virulent character of two different strains of PRRS viruses.

At least 10 gilts per group are included in each trial, which are derived from a PRRS free farm.

Animals are tested free of PRRS virus specific serum antibodies and negative for PRRSV. All animals included in the trial are of the same source and breed. The allocation of the animals to the groups is randomized.

Challenge is performed at day 90 of pregnancy with intranasal application of 1 ml PRRSV with 10⁵ TDCID₅₀ (third passage) per nostril. There are at least three groups for each test setup:

One group for ATCC VR-2332 challenge; one test group for challenge with the possibly attenuated virus; and one strict control group.

The study is deemed valid when the strict controls stay PRRS negative over the time course of the study and at least 25% less life healthy piglets are born in the ATCC VR-2332 challenged group compared to the strict controls.

Attenuation, in other words less virulence, is defined as the statistical significant change of one or more parameters determining reproductive performance:

Significant reduction in at least one of the following parameters for the test group (possibly attenuated virus) compared to the ATCC VR-2332 infected group is preferred:

frequency of stillborns

abortion at or before day 112 of pregnancy

number of mummified piglets

number of less live and weak piglets

preweaning mortality or furthermore a significant increase in one of the following parameters for the test group compared to the ATCC VR-2332 infected group is preferred:

number of piglets weaned per sow

number of live healthy piglets born per sow.

what is claimed is:

1. A live attenuated PRRS virus comprising ORF 1a , 1b and 2 essentially as in ATCC VR-2332, wherein said virus is not ATCC VR-2495, and wherein at least one of the amino acids in position 321 to 341 encoded by ORF 1a is not identical to the amino acid(s) of ATCC VR 2332 at the corresponding position(s) and/or at least one of the amino acids in position 936 to 956 encoded by ORF 1b is not identical to the amino acid(s) of the strain VR 2332 at the corresponding position(s) and/or at least one of the amino acids in position 1 to 20 encoded by ORF 2 is not identical to the amino acid(s) of ATCC VR 2332 at the corresponding position(s), and wherein said live attenuated PRRS virus is less virulent than ATCC VR-2332.

2. The live attenuated PRRS virus according to claim 1, wherein at least one of the amino acids in position 321 to 341 encoded by ORF 1a is deleted and/or at least one of the amino acids in position 936 to 956 encoded by ORF 1b is deleted and/or at least one of the amino acids in position 1 to 20 encoded by ORF 2 is deleted.

3. The live attenuated PRRS virus according to claim 1, wherein the amino acid in position 331 encoded by ORF 1a and/or the amino acid in position 946 encoded by ORF 1b and/or the amino acid in position 10 encoded by ORF 2 is/are not identical to the amino acid(s) of ATCC VR-2332 at the corresponding position(s).

4. The live attenuated PRRS virus according to claim 2 wherein the amino acid in position 331 encoded by ORF 1a and/or the amino acid in position 946 encoded by ORF 1b and/or the amino acid in position 10 encoded by ORF 2 is/are deleted.

5. A nucleotide sequence coding for a virus according to any one of claims 1 to 4.

6. The nucleotide sequence according to claim 5, wherein the nucleotide sequence has been modified to encode a virulence marker and/or a serological marker.

7. The nucleotide sequence according to claim 6, wherein the nucleic acid encoding said marker is located within any of the open reading frames encoding structural viral proteins.

8. A method for the generation of an infectious live attenuated PRRS virus comprising producing a recombinant nucleic acid comprising at least one full-length DNA copy or in vitro-transcribed RNA copy or a derivative of either wherein said nucleotide sequence is a nucleotide sequence according to claim 5.

9. A method for the generation of an infectious live attenuated PRRS virus comprising producing a recombinant nucleic acid comprising at least one full-length DNA copy or in vitro-transcribed RNA copy or a derivative of either wherein said nucleotide sequence is a nucleotide sequence according to claim 6.

10. The method according to claim 8, comprising specifically mutating the nucleotide sequence corresponding to the nucleotide sequence encoding amino acid positions 321 to 341 of ORF 1a and/or the nucleotide sequence corresponding to the nucleotide sequence encoding amino acid positions 936 to 956 of ORF 1b and/or the nucleotide sequence corresponding to the nucleotide sequence encoding amino acid positions 1 to 20 of ORF 2 wherein at least one nucleotide at said positions is substituted or deleted.

11. The method according to claim 9, comprising specifically mutating the nucleotide sequence corresponding to the nucleotide sequence encoding amino acid positions 321 to 341 of ORF 1a and/or the nucleotide sequence corresponding to the nucleotide sequence encoding amino acid positions 936 to 956 of ORF 1b and/or the nucleotide sequence corresponding to the nucleotide sequence encoding amino acid positions 1 to 20 of ORF 2 wherein at least one nucleotide at said positions is substituted or deleted

12. A pharmaceutical composition comprising a PRRS virus according to any one of claims 1 to 4; and a pharmaceutically acceptable carrier.

13. A method for the prophylaxis and treatment of PRRS infections comprising administering a PRRS virus according to any one of claims 1 to 4.


SEQUENCE LISTING (6 sequences)

SEQ ID NO: 1 - protein, 2502 aa, Porcine reproductive and respiratory syndrome virus
MSGILDRCTCTPNARVFMAEGQVYCTRCLSARSLLPLNLQVSELGVLGLFYRPEEPLRWTLPRAFPTVECSPAGACWLSAIFPIARMTSGNLNFQQRMVRVAAELYRAGQLTPAVLKALQVYERGCRWYPIVGPVPGVAVFANSLHVSDKPFPGATHVLTNLPLPQRPKPEDFCPFECAMATVYDIGHDAVMYVAERKVSWAPRGGDEVKFEAVPGELKLIANRLRTSFPPHHTVDMSKFAFTAPGCGVSMRVERQHGCLPADTVPEGNCWWSLFDLLPLEVQNKEIRHANQFGYQTKHGVSGKYLQRRLQVNGLRAVTDLNGPIVVQYFSVKESWIRHLKLAGEPSYSGFEDLLRIRVEPNTSPLADKEEKIFRFGSHKWYGAGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTSFDWDVVLPGVEAATQTIKLPQVNQCRALVPVVTQKSLDNNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQAICTLDGRLKFLPKMILETPPPYPCEFVMMPHTPAPSVGAESDLTIGSVATEDVPRILEKIENVGEMANQGPLAFSEDKPVDDQLVNDPRISSRRPDESTSAPSAGTGGAGSFTDLPPSDGADADGGGPFRTVKRKAERLFDQLSRQVFDLVSHLPVFFSRLFYPGGGYSPGDWGFAAFTLLCLFLCYSYPAFGIAPLLGVFSGSSRRVRMGVFGCWLAFAVGLFKPVSDPVGAACEFDSPECRNILHSFELLKPWDPVRSLVVGPVGLGLAILGRLLGGARCIWHFLLRLGIVADCILAGAYVLSQGRCKKCWGSCIRTAPNEVAFNVFPFTRATRSSLIDLCDRFCAPKGMDPIFLATGWRGCWAGRSPIEQPSEKPIAFAQLDEKKITARTVVAQPYDPNQAVKCLRVLQSGGRWWLSGPKSGQGFRCSIPSPFFPTGVKVDPDCRVVVDPDTFTAALRSGYSTTNLVLGVGDFAQLNGLKIRQISKPSGGGPHLMAALHVACSMALHMLAGIYVTAVGSCGTGTNDPWCANPFAVPGYGPGSLCTSRLCISQHGLTLPLTALVAGFGIQEIALVVLIFVSIGGMAHRLSCKADMLCVLLAIASYVWVPLTWLLCVFPCWLRCFSLHPLTILWLVFFLISVNMPSGILAMVLLVSLWLLGRYTNVAGLVTPYDIHHYTSGPRGVAALATAPDGTYLAAVRRAALTGRTMLFTPSQLGSLLEGAFRTRKPSLNTVNVIGSSMGSGGVFTIDGKVKCVTAAHVLTGNSARVSGVGFNQMLDFDVKGDFAIADCPNWQGAAPKTQFCTDGWTGRAYWLTSSGVEPGVIGKGFAFCFTACGDSGSPVITEAGELVGVHTGSNKQGGGIVTRPSGQFCNVAPIKLSELSEFFAGPKVPLGDVKVGSHIIKDISEVPSDLCALLAAKPELEGGLSTVQLLCVFFLLWRMMGHAWTPLVAVSFFILNEVLPAVLVRSVFSFGMFVLSWLTPWSAQVLMIRLLTAALNRNRWSLAFFSLGAVTGFVADLAATQGHPLQAVMNLSTYAFLPRMMVVTSPVPVITCGVVHLLAIILYLFKYRGPHHILVGDGVFSAAFFLRYFAEGKLREGVSQSCGMNHESLTGALAMRLNDEDLDFLMKWTDFKCFVSASNMRNAAGQFIEAAYAKALRVELAQLVQVDKVRGTLAKLEAFADTVAPQLSPGDIVVALGHTPVGSIFDLKVGSTKHTLQAIETRVLAGSKMTVARVVDPTPTPPPAPVPIPLPPKVLENGPNAWGDEDRLNKKKRRRMEALGIYVMGGKKYQKFWDKNSGDVFYEEVHNNTDEWECLRVGDPADFDPEKGTLCGHVTIENKAYHVYTSPSGKKFLVPVNPENGRVQWEAAKLSVEQALGMMNVDGELTAKELEKLKRIIDKLQGLTKEQCLNC

SEQ ID NO: 2 - protein, 1457 aa, Porcine reproductive and respiratory syndrome virus
LAASDLTRCGRGGLVVTETAVKIVKFHNRTFTLGPVNLKVASEVELKDAVEHNQHPVARPIDGGVVLLRSAVPSLIDVLISGADASPKLLAHHGPGNTGIDGTLWDFESEATKEEVALSAQIIQACDIRRGDAPEIGLPYKLYPVRGNPERVKGVLQNTRFGDIPYKTPSDTGSPVHAAACLTPNATPVTDGRSVLATTMPPGFELYVPTIPASVLDYLDSRPDCPKQLTEHGCEDAALKDLSKYDLSTQGFVLPGVLRLVRKYLFAHVGKCPPVHRPSTYPAKNSMAGINGNRFPTKDIQSVPEIDVLCAQAVRENWQTVTPCTLKKQYCGKKKTRTILGTNNFIALAHRAVLSGVTQGFMKKAFNSPIALGKNKFKELQTPVLGRCLEADLASCDRSTPAIVRWFAANLLYELACAEEHLPSYVLNCCHDLLVTQSGAVTKRGGLSSGDPITSVSNTIYSLVIYAQHMVLSYFKSGHPHGLLFLQDQLKFEDMLKVQPLIVYSDDLVLYAESPTMPNYHWWVEHLNLMLGFQTDPKKTAITDSPSFLGCRIINGRQLVPNRDRILAALAYHMKASNVSEYYASAAAILMDSCACLEYDPEWFEELVVGIAQCARKDGYSFPGTPFFMSMWEKLRSNYEGKKSRVCGYCGAPAPYATACGLDVCIYHTHFHQHCPVTIWCGHPAGSGSCSECKSPVGKGTSPLDEVLEQVPYKPPRTVIMHVEQGLTPLDPGRYQTRRGLVSVRRGIRGNEVGLPDGDYASTALLPTCKEINMVAVASNVLRSRFIIGPPGAGKTYWLLQQVQDGDVIYTPTHQTMLDMIRALGTCRFNVPAGTTLQFPVPSRTGPWVRILAGGWCPGKNSFLDEAAYCNHLDVLRLLSKTTLTCLGDFKQLHPVGFDSHCYVFDIMPQTQLKTIWRFGQNICDAIQPDYRDKLMSMVNTTRVTYVEKPVRYGQVLTPYHRDREDDAITIDSSQGATFDVVTLHLPTKDSLNRQRALVAITRARHAIFVYDPHRQLQGLFDLPAKGTPVNLAVHCDGQLIVLDRNNKECTVAQALGNGDKFRATDKRVVDSLRAICADLEGSSSPLPKVAHNLGFYFSPDLTQFAKLPVELAPHWPVVSTQNNEKWPDRLVASLRPIHKYSRACIGAGYMVGPSVFLGTPGVVSYYLTKFVKGGAQVLPETVFSTGRIEVDCREYLDDREREVAASLPHGFIGDVKGTTVGGCHHVTSRYLPRVLPKESVAVVGVSSPGKAAKALCTLTDVYLPDLEAYLHPETQSKCWKMMLDFKEVRLMVWKDKTAYFQLEGRYFTWYQLASYASYIRVPVNSTVYLDPCMGPALCNRRVVGSTHWGADLAVTPYDYGAKIILSSAYHGEMPPGYKILACAEFSLDDPVKYKHTWGFESDTAYLYEFTGNGEDWEDYNDAFRARQEGKIYKATATSLKFYFPPGPVIEPTLGLN

SEQ ID NO: 3 - protein, 256 aa, Porcine reproductive and respiratory syndrome virus
MKWGPCKAFLTKLANFLWMLSRSSWCPLLISLYFWPFCLASPSPVGWWSFASDWFAPRYSVRALPFTLSNYRRSYEAFLSQCQVDIPTWGTKHPLGMLWHHKVSTLIDEMVSRRMYRIMEKAGQAAWKQVVSEATLSRISSLDVVAHFQHLAAIEAETCKYLASRLPMLHNLRMTGSNVTIVYNSTLNQVFAIFPTPGSRPKLHDFQQWLIAVHSSIFSSVAASCTLFVVLWLRVPILRTVFGFRWLGAIFLSNSQ

SEQ ID NO: 4 - DNA, 7509 bp, Porcine reproductive and respiratory syndrome virus
atgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgtccctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaagtctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacctaaacggacctatcgtcgtacagtacttctccgttaaggagagttggatccgccatttgaaactggcgggagaacccagctactctgggtttgaggacctcctcagaataagggttgagcctaacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcacagcggagggaatcaccctgaccaagtgcgcttagggaaaattatcagcctttgtcaggtgattgaggactgctgctgttcccagaacaaaaccaaccgggtcaccccggaggaggtcgcagcaaagattgacctgtacctccgtggtgcaacaaatcttgaagaatgcttggccaggcttgagaaagcgcgcccgccacgcgtaatcgacacctcctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaactactactaccgtgcgcaaggtgacgaagttcgtcaccgtgaaagactaaccgccgtgctctccaagttggaaaaggttgttcgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccaggacgagcccctggatttgtctgcttcctcacagactgaatatgaggcctctcccccagcaccgccgcagagcgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaaacatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggctcttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctaccctggcggtggttattctccgggtgattggggttttgcagcttttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgttgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcagtcgggtgggcgatggtggctaagcggtcccaaaagtggtcaaggtttccgctgttccattccgagccccttctttcccactggagtgaaagttgaccctgattgcagggtcgtggttgaccctgacactttcactgcagctctccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccaggttgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctcaccatcctatggttggtgtttttcttgatttctgtgaatatgccttcaggaatcttggccatggtgttgttggtttctctttggcttcttggtcgttatactaatgttgctggccttgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatagctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctatcctggctcacgccatggtctgcgcaagttctgatgatcaggcttctgacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcccgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtatcttcgacctaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcaatgggaagctgcaaagctttccgtggagcaggccctaggtatgatgaatgtcgacggcgaactgactgccaaagaactggagaaactgaaaagaataattgacaaactccagggcctgactaaggagcagtgtttaaactgctag

SEQ ID NO: 5 - DNA, 4374 bp, Porcine reproductive and respiratory syndrome virus
ctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggagttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacctggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaaaggcacaagccctttagacgaggtgctggaacaagtcccgtataagcccccacggaccgttatcatgcatgtggagcagggtctcaccccccttgatccaggtagataccaaactcgccgcggattagtctctgtcaggcgtggaattaggggaaatgaagttggactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggtccgtgggttcgcatcctagccggcggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtggttacattgcatttgcccactaaagattcactcaacaggcaaagagcccttgttgctatcaccagggcaagacacgctatctttgtgtatgacccacacaggcagctgcagggcttgtttgatcttcctgcaaaaggcacgcccgtcaacctcgcagtgcactgcgacgggcagctgatcgtgctggatagaaataacaaagaatgcacggttgctcaggctctaggcaacggggataaatttagggccacagacaagcgtgttgtagattctctccgcgccatttgtgctgatctagaagggtcgagctctccgctccccaaggtcgcacacaacttgggattttatttctcacctgatttaacacagtttgctaaactcccagtagaacttgcacctcactggcccgtggtgtcaacccagaacaatgaaaagtggccggatcggctggttgccagccttcgccctatccataaatacagccgcgcgtgcatcggtgccggctatatggtgggcccttcggtgtttctaggcactcctggggtcgtgtcatactatctcacaaaatttgttaagggcggggctcaagtgcttccggagacggttttcagcaccggccgaattgaggtagactgccgggaatatcttgatgatcgggagcgagaagttgctgcgtccctcccacacggtttcattggcgacgtcaaaggcactaccgttggaggatgtcatcatgtcacctccagatacctcccgcgcgtccttcccaaggaatcagttgcggtagtcggggtttcaagccccggaaaagccgcgaaagcattgtgcacactgacagatgtgtacctcccagatcttgaagcctatctccacccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcccgtcaactctacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttataaggccactgccaccagcttgaagttttattttcccccgggccctgtcattgaaccaactttaggcctgaattga

SEQ ID NO: 6 - DNA, 771 bp, Porcine reproductive and respiratory syndrome virus
atgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggatgctttggcaccataaggtgtcaaccctgattgatgaaatggtgtcgcgtcgaatgtaccgcatcatggaaaaagcagggcaggctgcctggaaacaggtggtgagcgaggctacgctgtctcgcattagtagtttggatgtggtggctcattttcagcatctagccgccattgaagccgagacctgtaaatatttggcctcccggctgcccatgctacacaacctgcgcatgacagggtcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtga